(19) United States
(12) Patent Application Publication
(10) Pub. No.: US 2009/0216207 A1
(43) Pub. Date: Aug. 27, 2009

(54) Biodegradable Barrier Film

(76) Inventor: Soeren Kirkegaard Nielsen, Faaborg (DK)

Correspondence Address: JACOBSON HOLMAN PLLC, 400 SEVENTH STREET N.W., SUITE 600, WASHINGTON, DC, 20004, US

(21) Appl. No.: 12/223,711

(22) PCT Filed: Feb. 9, 2007
(86) PCT No.: PCT/DK2007/050017

§ 371 Date: Jan. 12, 2009

(30) Foreign Application Priority Data
Feb. 10, 2006 (DK) PA 2006 00195

(51) Int. Cl. A61F 5/441; B32B 5/16

(52) U.S. Cl. 604/328; 604/333; 428/220; 428/331

(57) ABSTRACT

The present invention discloses the construction of a biodegradable film that has barrier towards faeces odour and with no cracking sounds. This is obtained through using a substantially exfoliated silicate filler material, which is applied with water (evaporating under mild conditions) such that the softener of the biodegradable material can be maintained in the film.

### FIELD OF THE INVENTION

The present invention relates to a film suitable for use as an ostomy bag.

### BACKGROUND

In connection with surgery for a number of diseases in the gastrointestinal tract a consequence is, in many cases, that the colon, the ileum or the urethra has been exposed surgically and the patient is left with an abdominal stoma and the effluents or waste products of the body, which are conveyed through these organs, are discharged through the artificial orifice or opening and are collected in a collection bag, which is usually adhered to the skin by means of an adhesive wafer or plate having an inlet opening for accommodating the stoma. Also, in connection with a fistula, the patient will have to rely on an appliance to collect the bodily material emerging from such opening.

When replacing the ostomy bag, it would be advantageous for an ostomy-operated patient if the used ostomy bag could be w.c. disposable instead of having to go with the day refuse, with odour problems as a consequence. However, to make such flushing of the used bag environmentally friendly it is required that the bag is biodegradable.

EP0703762 describes a so-called bag-in-bag solution. Here, the outer bag is similar to a conventional ostomy bag with charcoal filter. This outer bag provides the barrier towards odour. The inner bag, which is the flushable one, is biodegradable and or/water soluble. The two bags are separated before the inner bag is flushed out in the w.c.

GB2083762 describes a film made of PVOH coated with PVDC, vinyl chloride-vinylidene chloride-copolymer, atactic polypropylene, nitrocellulose, waxes, greases, silicones, or pressure-sensitive adhesives. The principle in a multi-layer laminate is that a biodegradable and or water-soluble layer gives the main part of the mechanical strength, and a mechanical weak layer gives the main part of the barrier properties.

In the course of time several experiments have been made to prepare flushable ostomy bags, but the preparation of a foil that is both biodegradable and at the same time has the sufficient barrier towards faeces odour has not succeeded until date.

### SUMMARY OF THE INVENTION

The present invention discloses the construction of a biodegradable film that has barrier towards faeces odour and with no cracking sounds.

### DETAILED DISCLOSURE

A central aspect of the present invention relates to a film of biodegradable material comprising on at least one surface a substantially exfoliated silicate filler material.

This film meets the following requirements:
1) It is biodegradable, i.e. 90 percentage of the weight of the film is degraded after six months (as determined by ISO 14852);
2) It is impervious to water and faeces odour for at least for 10 hours; examples 2 and 3 illustrates a marked reduction in oxygen permeability and smell penetration when a biodegradable film is coated with a substantially exfoliated silicate filler material.
3) It is at least, or less, stiff and crackling than a standard ostomy bag; example 4 illustrates that a biodegradable material (with no crackling) still has no crackling after coating.

In preferred embodiments, the film also is heat-sealed and is transparent.

Hereby a dilemma in the art is solved: Laminates of polymers cannot both be sufficiently impermeable for odour and at the same time be biodegradable; and Glass and metal can only be vapour-deposited on polymers that do not contain softeners, as the softener evaporates during the vacuum process. Biodegradable polymers without softeners are to stiff and crackling to be useable as ostomy bags. By using a substantially exfoliated silicate filler material, e.g. Nanolok®, which is applied with water, which evaporates under mild conditions, the softener can be maintained in the film.

In one aspect of the invention the biodegradable material is degraded 90% (of weight) after six months. It is, however, preferred that the biodegradable material is degraded 100% after six months, allowing for 10% of the weight of the film to be non-degradable material (e.g. the coating of substantially exfoliated silicate filler material). In a preferred embodiment, the biodegradable material is selected among biodegradable polyesters in general. Particularly preferred is the biodegradable material selected from the group consisting of PVOH (polyvinyl alcohol), Starch, PLA (polylatic acid), PHB (polyhydroxybutyrate), and Polycaprolactone. Examples of such films are Ecoflex (an aliphatic-aromatic copolyester based on the monomers 1,4-butanediol, adipic acid and terephthalic acid), Mater-Bi (a blend of starch and Ecoflex), and PVOH.

The biodegradable film can be softened e.g. with glycerol which is 100% biodegradable.

In the present invention, a “film” is understood as a thin, planar sheet with a thickness of about 10-100 micrometer. Typically, for use as an ostomy bag, the dimensions (height×with) is in the range of 126×200 mm.

By exfoliated means the complete separation of individual layers of the original particle, so that polymer completely surrounds each particle. desirably so much polymer is present between each platelet, that the platelets are randomly spaced. No X-ray line appears because of the random spacing of exfoliated platelets. Often exfoliated is used to describe a surface treated nano-clay, which possesses a sufficiently enlarged gallery spacing to permit the nano-clay to fully disperse (exfoliate) in a plastic matrix.

Various methods exist to apply the substantially exfoliated silicate filler material to the biodegradable material. In one embodiment, the exfoliated silica filler material is coated on the surface of the biodegradable material.

In one embodiment the substantially exfoliated silicate filler material is exfoliated silicate nano-flake material. In another embodiment the substantially exfoliated silicate filler material is exfoliated silicate flake filler material.

The coating may consist of nanoclay or any other mineral material having the ability to be intercalated, exfoliated, and dispersed in a liquid suspension. The suspension may also contain a polymer adding cohesion to the coating after the liquid is evaporated. In order for the coating to function as a gas barrier the clay particles must have one dimension in the nanometer range, preferably they are bigger in dimensions being parallel to the substrate. An preferred nanoclay is nanoclay from the smectite family.

In a preferred embodiment the exfoliated silica filler material is (vermiculite e.g. the microlite®) as described in WO05044938 and WO05063871.

Nanolok is an aqueous suspension of silica nano-flakes and polymers (see http://www.inmat.com/tech.packagings.html). After Nanolok has been coated onto the substrate (the biodegradable film) the water evaporates, resulting in a layer of nano-flakes and polymers. This layer is very impermeable (O₂-permeability <0.01 CC×mil/100 in²×day×atm), a factor ten (closer) than PVDC. Due to this, the Nanolok layer can be made very thin, 1-2 μm, as thin that it constitutes less than 10% of the complete film, which together can be seen as biodegradable.

This solution is advantageous, as you don't need to separate bags before flushing the inner bag, and keep the (somewhat soiled) outer bag.

One aspect of the invention relates to the use of a film of biodegradable material coated with a substantially exfoliated silicate filler material for the manufacture of an ostomy bag.

Another aspect of the invention relates to a method for manufacturing an ostomy bag comprising the steps of:
(a) providing a film of biodegradable material
(b) coat the film of (a) with a substantially exfoliated silicate filler material.
   The film can also be used in other contexts, where biodegradability/solubility shall be combined with a high barrier, for instance for urine bags, but also in completely other connections where a product is to be sealed, for instance for packaging of foodstuffs or where a controlled release is desired, i.e. the packaging degrades/dissolves whereby the content is released.

### EXAMPLES

### Example 1

Flush test with two narrow Toilet's (tube dim. Ø73-80 mm): 3 L/6 L Gustav Berg toilet, and 4 L/6 L lfö cera toilet.

To obtain good flushability, it is important to have a flexible film. The maximum stiffness that allowed full flushability was the Materbi 50 my biofilm. If this stiffness is exceeded, the ability to fold up around the contents is decreased, thereby decreasing flow through the toilet tube.

The preferred size of the bag is (with×height) 126×200 mm.

### CONCLUSION

The flush pouch with these dimensions can be flushed without complications with only one flush×4 L. (flush with only 3 L will in some cases need two flushings).

### Example 2

### OTR Test

The following films were evaluated on a Mocon 2/20 Ox Tran oxygen analyzer at 0% relative humidity and 23° C. The films were then coated with Nanolok PT 3575 using a #12 meyer rod and air dried. The films were tested under the same conditions and the results are listed below:

| Substrate | Thickness^{b} | Coating | OTR^{a} |
|---|---|---|---|
| A - blend of starch, | 90 | None | 247 |
| PLA & glycerol | | | 238 |
| B - pure PLA | 26 | None | 655 |
| | | | 695 |
| C - Biodegradable | 35 | None | 1721 |
| polyester | | | 1640 |
| A - blend of starch, | 90 | Nanolok PT 3575 modified | 2.6 |
| PLA & glycerol | | | 2.3 |
| B - pure PLA | 26 | Nanolok PT 3575 | 2.0 |
| | | | 1.8 |
| C - Biodegradable | 35 | Nanolok PT 3575 modified | 5.0 |
| polyester | | | 13.7 |

^{a}Oxygen transmission rate (OTR) is in units of cc/m² day atm @ 23° C., 0% RH.
^{b}Thickness in microns.

The oxygen transmission rate for films A and B were reduced significantly from 200-600 down to 2-2.5 cc/m2 day atm@23° C., 0% RH. The oxygen transmission rate was also significantly reduced for film C. However, using a meyer rod was not perfect for a film with such high flexibility, and it is speculated that the coating obtained was not even. Even lower OTR's (higher reduction) is anticipated with even coating on this type of film.

### Example 3

### Scatol Test

A few drops of scatol (3-Methylindol) is applied to a beaker and the test-film set on top. The beaker is placed within a larger beaker with a screw-lid. The test-persons unscrews the lid and samples the scatol small on a scale from 1 (no smell) to 5 (intense smell). The results are presented below:

| | Time from closure of scatol beaker | | | | | | | |
|---|---|---|---|---|---|---|---|---|
| Test-film | ½ | 1½ | 2½ | 3½ | 4½ | 5½ | 6½ | 24 |
| Mater-Bi | 1 | 1 | 2 | 3 | 2 | 4 | 3 | 5 |
| Mater-Bi | 1 | 1 | 1 | 3 | 2 | 4 | 4 | 5 |
| Mater-Bi | 1 | 1 | 1 | 4 | 2 | 5 | 4 | 5 |
| Mater-Bi | 1 | 1 | 1 | 3 | 2 | 3 | 2 | 5 |
| Average | 1 | 1 | 1.3 | 3.3 | 2 | 4 | 3.3 | 5 |
| Mater-Bi + coating | 1 | 1 | 1 | 1 | 1 | 1 | 2 | 3 |
| Mater-Bi + coating | 1 | 1 | 1 | 1 | 1 | 2 | 2 | 2 |
| Mater-Bi + coating | 1 | 1 | 1 | 1 | 1 | 1.5 | 2 | 1 |
| Mater-Bi + coating | 1 | 1 | 1 | 1 | 1.5 | 3 | 2 | 2 |
| Average | 1 | 1 | 1 | 1 | 1.1 | 1.9 | 2 | 2 |
| PVDC | 1 | 1 | 1 | 1 | 1 | 1 | 2 | 2 |
| PVDC | 1 | 1 | 1 | 1 | 1 | 1 | 2 | 1 |
| PVDC | 1 | 1 | 1 | 1 | 1 | 1 | 1 | 1 |
| Average | 1 | 1 | 1 | 1 | 1 | 1 | 1.7 | 1.3 |

The smell penetrates the Mater-Bi film after 3½ hours. Penetrations is when the subjective smell is >1. When coated, the smell is contained for up to 6½ hours before smell penetration. The variability in penetration indicates that the coating is not uniform - yet.

### Example 4

### Crackling Test

A panel of 4 persons were asked to evaluate the level of crackling. However, they could not determine any difference between the samples of Mater-Bi with coating (Nanolok), Mater-Bi without coating and two standard ostomy bags presently on the market.

It is concluded that the coating does not cause crackling.

1. Film of biodegradable material comprising on one surface a substantially exfoliated silicate filler material.

2. Film according to claim 1, wherein the biodegradable material is degraded 90% (of weight) after six months.

3. Film according to claim 1, wherein the biodegradable material is selected from the group consisting of PVOH (polyvinyl alcohol), Starch, PLA (polylatic acid), PHB (polyhydroxybutyrate), and Polycaprolactone.

4. Film according to claim 1, wherein the film has a thickness of 10 to 100 micrometer.

5. Film according to claim 1, wherein the substantially exfoliated silicate filler material is coated on the surface of the biodegradable material.

6. Use of a film of biodegradable material coated with a substantially exfoliated silicate filler material for the manufacture of an ostomy bag.

7. Method for manufacturing an ostomy bag comprising the steps of:
(a) providing a film of biodegradable material
(b) coat the film of (a) with a substantially exfoliated silicate filler material.

8. Ostomy bag comprising a film according to claim 1.

9. Urine bag comprising a film according to claim 1.
